(19) United States
(12) Patent Application Publication
(10) Pub. No.: US 2008/0118477 A1
(43) Pub. Date: May 22, 2008

(54) UMBILICAL CORD MESENCHYMAL STEM CELLS SUPPORT CORD BLOOD HEMATOPOIESIS

(75) Inventor: Kent W. Christopherson, Aurora, IL (US)

Correspondence Address: BARNES & THORNBURG LLP, P.O. BOX 2786, CHICAGO, IL, 60690-2786, US

(73) Assignee: RUSH UNIVERSITY MEDICAL CENTER, Chicago, IL (US)

(21) Appl. No.: 11/937,605

(22) Filed: Nov. 9, 2007

Related U.S. Application Data
(60) Provisional application No. 60/865,066, filed on Nov. 9, 2006.

(51) Int. Cl. A61K 35/48; C12N 5/00; C12N 5/06

(52) U.S. Cl. 424/93.7; 435/373; 435/366

(57) ABSTRACT

Hematopoietic stem cells (HSC) are routinely obtained from bone marrow, mobilized peripheral blood, and umbilical cord blood. Traditionally, bone marrow has been utilized as a source of mesenchymal stem cells (MSC). The use and expansion of umbilical cord MSC to support the growth, viability, and maintenance of cord blood derived HSC, during clonal expansion and during differentiation is demonstrated. Umbilical cord derived MSC and cord blood derived HSC are genetically matched, to improve overall expansion and utility of low volume cord blood HSC. Finally, umbilical cord MSC derived from Wharton's jelly and grown with genetically matched cord blood HSC can be used as a cellular therapeutic in the transplant setting for the treatment of malignant and non-malignant hematologic diseases.

This application claims priority to U.S. Ser. No. 60/865,066 filed Nov. 9, 2006.

### BACKGROUND

Methods and compositions are described to isolate mesenchymal stem cells (MSC) from Wharton's jelly, the mucous connective tissue of human umbilical cord, and to combine the isolated MSC with hematopoietic stem cells (HSC), in particular from cord blood, for improved expansion and survivability of the hematopoietic stem cells. Use of combinations of HSC and MSC for treatment of hematologic diseases is also described.

Hematopoietic stem cells (HSC) are routinely obtained from bone marrow, peripheral blood, and umbilical cord blood. Traditionally, adult bone marrow has been utilized as a source of mesenchymal stem cells (MSC) but aspirating bone marrow from the patient is an invasive and painful procedure. Bone marrow-derived MSC (BM-MSC) have been reported to maintain the growth of HSC obtained from cord blood and have been utilized for cord blood expansion purposes. Co-culture of HSC with BM-MSC has also been reported to promote engraftment of CD34⁺ defined cord blood hematopoietic stem and progenitor cells (HSC/HPC) into nonobese diabetic/severe combined immunodeficient (NOD/SCID) mice. However, the use of a BM-MSC as a feeder layer to support the long term culture of cord blood HSC is not ideal for clinical transplants.

Mesenchymal stem cells have a unique ability as multipotent progenitors capable of supporting hematopoiesis and differentiating into multiple lineages (osteogenic, adipogenic, and chondrogenic, myogenic, cardiomyogenic, and the like) if the cells are cultured under specific conditions. MSC are thus useful in tissue engineering and cell-based therapy. Mesenchymal stem cells are a rare population including approximately 0.001% to 0.01% of adult human bone marrow. Further, the number and the differentiating potential of bone marrow MSC decreases with age. Therefore, the search for alternative sources of MSC is of significant value.

Controversy exists as to whether cord blood can serve as a source of sufficient numbers of MSC for clinical use. The recent isolation of MSC from a novel source, the Wharton's jelly of umbilical cord segments, has demonstrated that a viable population of MSC can come from a term umbilical cord although in very low numbers. Wharton's jelly is the primitive connective tissue of the human umbilical cord and was first described by Thomas Wharton in 1656. The umbilical cord has two arteries and one vein embedded in Wharton's jelly, a loose myxoematus tissue of mesodermal origin. This jelly acts as a physical buffer and prevents kinking of the cord and interference of maternal-fetal circulation.

Isolation of fibroblast-like cells from the Wharton's jelly of the umbilical cord was originally described in 1991. More recently, putative MSC have been reported from the umbilical cord itself using two different dissection methods, (1) from the subendothelial layer of the cord vein, or (2) from the Wharton's jelly. There are reports of isolated MSC-like cells, matrix cells, and human umbilical cord perivascular cells as sources for mesenchymal progenitors, and mesenchymal stem cells differentiation into nerve-like cells. However none of the reports demonstrate or claim to have isolated MSC that support hematopoiesis, which to a hematologist is the only functional definition that matters since this is the primary function of MSC in the bone marrow and the only definition that matters for hematopoietic stem cell transplantation.

Chondrogenic progenitor cells were isolated from Wharton's jelly by removing blood and blood vessels from human umbilical cord and incubating the remaining tissue under conditions purported to allow the prechondrocytes to proliferate. As such, the method did not distinguish different cell types present in Wharton's jelly, but rather relied on migration from the tissue, or selecting growth conditions favoring prechondrocytes. Although the prechondrocytes appeared to be a mixed cell population, removal of cord blood suggested that the mixed population was solely from the Wharton's jelly and derived from cord blood progenitor cells.

Transplantation of porcine umbilical cord matrix cells into rat brain was investigated. Two distinct populations were obtained—spherical and flat mesenchymal cells. No co-culture with HSC was described. The cells were further genetically modified for transplantation. The cells did not appear to stimulate immune rejection when implanted cross-species.

Term placenta has been identified as a possible source for HSC. Umbilical cord blood has also been accepted as a source for hematopoietic stem cells although in a very low yield.

Hematopoietic stem cell transplantation (HSCT) is a successful treatment option for many patients with severe hematologic diseases. Current transplant protocols require transplantation of large numbers of hematopoietic stem and progenitor cells (HSC/HPC) in order to overcome the inherently inefficient transplant process and achieve successful reconstitution of the hematopoietic system. Under current practice, the necessary HSC have been traditionally obtained by aspirating a large volume of bone marrow (BM) from a donor or by apherisis procedures following growth factor-induced mobilization of peripheral blood stem cells (PBSC). Alternatively, umbilical cord blood (CB) that is collected at the time of a baby's birth can be utilized as a donor source of HSC for transplantation into child recipients. CB is an alternative to BM when a traditional matched BM donor is not available. However, patient survival is compromised when large numbers of HSC are needed and donor HSC numbers are limited. This is the case when CB is utilized as a donor source for transplantation into adult recipients.

For transplantation, HSC and MSC would ideally be obtained from the same donor source, or at least HLA matched, as well as matched with the recipient—thereby eliminating the potential for complications resulting from a HSC and MSC mismatch. Alternatively, there may be an advantage to obtaining HLA-matched donor MSC from a non-adult tissue source such as cold blood. However, the numbers of MSC obtainable from cord blood are small in comparison to bone marrow.

Existing technologies are based on mesenchymal stem cells derived from the bone marrow. In this situation, it is not possible to find a genetic match to cord blood derived hematopoietic stem cells for transplant purposes. In contrast, umbilical cord mesenchymal stem cells can be obtained from the same donor as cord blood hematopoietic stem cells and thereby allow the possibility of genetically matched transplant of both hematopoietic and mesenchymal stem cells as a cellular therapy.

### SUMMARY

UC-MSC were then tested for their ability to support the growth of pooled CD34⁺ cord blood cells in long term culture-initiating cell (LTC-IC) assays, as compared to BM-MSC. This information provides evidence that the cells isolated having fibroblast morphology were in fact able to behave in a manner consistent with MSC with respect to hematology. No cell isolated from the umbilical cord by any other previous methodology has been shown to have the ability to support hematopoietic stem and progenitor cell growth or differentiation in a manner consistent only with that of MSC, and that has been previously reported for BM-MSC. This finding is furthermore unexpected since there is no previous suggestion that there is a need for or the existence of cells in the Wharton's Jelly of the umbilical cord that act to provide support for HSC/HPC in a manner similar to that previously described for bone marrow derived marrow stromal cells, also known as mesenchymal stem cells.

Umbilical cord derived MSC was shown to support the long term in vitro growth, viability, and maintenance of cord blood derived HSC, during clonal expansion and during differentiation.

Umbilical cord derived MSC and cord blood derived HSC are genetically matched to improve the overall expansion and utility of low volume cord blood HSC.

Umbilical cord MSC derived from Wharton's jelly and grown with genetically matched cord blood HSC are used as a cellular therapeutic in the transplant setting for the treatment of malignant and non-malignant hematologic diseases.

Umbilical cord MSC grown in culture separately or in co-culture with cord blood HSC may be transplanted individually, consecutively, or sequentially with cord blood HSC as a cellular therapeutic for malignant and non-malignant hematologic diseases.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1. Umbilical Cord-Mesenchymal Stem Cells (UC-MSC). Adherent cells isolated from the Wharton's jelly of umbilical cord segments were cultured for extended periods of time and displayed morphology characteristic of mesenchymal stem cells. (A) Shown is a typical colony formed after 7 days in culture. These cells can be detached and re-plated to form a monolayer of cells. (B) Shown is a typical monolayer of UC-MSC (passage 5) that was subsequently utilized for experiments described herein.

FIG. 2. Flow Cytometric Analysis of Mesenchymal Stem Cells. Bone marrow-derived MSC (BM-MSC) and umbilical cord-derived MSC (UC-MSC) were phenotypically evaluated. MSC are expected to be positive for CD73, CD105, CD90, CD166, CD44, HLA-ABC, and CD29, and negative for CD45, CD34, CD117, CD38, and HLA-DR, based on the published literature. Although passage 3 (p3) UC-MSC do not strictly meet this definition, later passages do. It was observed that UC-MSC do display the typical expression of cell surface markers by passage 5. UC-MSC have an extensive ability to be passaged for an extended period of time. UC-MSC passage 13 (p13) UC-MSC are shown above.

FIG. 3. Long Term Culture-Initiating Cell (LTC-IC) Colony on UC-MSC. Umbilical cord-derived mesenchymal stem cells (UC-MSC) were observed to have the capacity to support the maintenance of hematopoietic colonies. (A) Shown is a typical colony formed from pooled human cord blood CD34⁺ cells grown on top of UC-MSC following 35 days of culture. These colonies can then be detached and re-plated in methylcellulose for colony formation. (B) Shown is a typical progenitor colony formed following 14 days of methylcellulose culture after having been previously cultured for 35 days on UC-MSC

FIG. 4. Long Term Culture-Initiating Cell Assays. After 35 days of culture on either bone marrow (BM)- or umbilical cord (UC)-derived mesenchymal stem cells (MSC) followed by 14 days of progenitor colony formation in methylcellulose media, the numbers of colony forming cells (CFC) per 1×10⁴ CD34⁺ cord blood cells were assessed. UC-MSC were observed to support cord blood LTC-IC formation at levels comparable to BM-MSC.

### DETAILED DESCRIPTION OF THE INVENTION

Cord blood has been used as a transplantable source of HSC/HPC since it was first used for hematopoietic stem cell transplantation (HSCT) in 1988. Given that cord blood is readily available, has a lower histocompatability requirement, and carries a reduced risk of graft vs. host disease, there are advantages to utilizing cord blood for allogeneic HSCT, especially when a matched bone marrow or peripheral blood stem cell donor is not available. However, the amount of cord blood collected is a limiting factor, in most cases only yielding sufficient quantities for a child recipient. This problem may be overcome in part through ex vivo expansion of the donor cell population in a manner that supports the maintenance of subsequent HSC homing and engraftment potential.

The use of MSC as a feeder layer is an attractive alternative to cytokine based ex vivo expansion, based on reports that have described the potential of MSC to promote engraftment of CD34⁺ HSC/HPC cells into NOD/SCID mice. UC-MSC, described by others putatively as fibroblast-like cells, candidate MSC-like cells, matrix cells, or human umbilical cord perivascular cells, were cultured.

These putative UC-MSC act as bona fide MSC in the context of supporting hematopoiesis. Results show that UC-MSC have the capacity to support long term maintenance of HSC, as defined by the LTC-IC assay. These findings have therapeutic applications with respect to ex vivo stem cell expansion of cord blood HSC utilizing a UC-MSC feeder layer. In addition, co-transplantation of matched mesenchymal and hematopoietic stem cells from the same umbilical cord and cord blood donor source or from HLA-matched umbilical cord and cord blood donors is contemplated.

Umbilical cord derived MSC (UC-MSC) support the long term growth of cord blood derived HSC ex vivo or in vivo. Phenotypically defined MSC were isolated from the Wharton's jelly of umbilical cord segments and defined morphologically and by cell surface markers. By dissecting out the veins and arteries of cord segments and exposing the Wharton's jelly, the cells of interest were accessed. The 3 cm cord segments were then placed in 20 ml of collagenase solution (1 mg/ml, Sigma) for 18 hrs at room temperature. After incubation, the remaining tissue was removed and the cell suspension was diluted with PBS into two 50 ml tubes and centrifuged. Cells were then washed in PBS and counted using hematocytometer. 5-20×10⁶ cells were then plated in a 6 cm tissue culture plate in low-glucose DMEM (Gibco) with 10% FBS (Hyclone), 2 mM L-Glutamine (Gibco), 100 U/ml penicillin/100 ug/ml streptomycin/0.025 ug/ml amphotericin B (Gibco). After 48 hrs cells were washed with PBS and given fresh media. Cells were given new media twice weekly. After 7 days, cells were approximately 70-80% confluent and were passed using HyQTase (Hyclone) into a 10 cm plate. Cells were then regularly passed 1:2 every 7 days or upon reaching 80% confluence.

Phenotypically defined CD34⁺ HSC were obtained from human CB in the following manner. Human umbilical CB samples were collected from the Labor and Delivery Units with Institutional Review Board (“IRB”) approvals. MNC were isolated by density centrifugation, and CD34 positive enrichment of a purity of greater than 98% is performed utilizing an AutoMACS (Miltenyi Biotech, Auburn, Calif.). Both total CB units and CD34⁺ CB cells were used in the in vitro assays.

Variations on an ex vivo co-culture system for the expansion of CB donor HSC on MSC were used. CD34⁺ enriched CB cells, as well as total nucleated cells (TNC) from CB were utilized for expansion studies and were referred to as donor units. UC-MSC were utilized for co-culture and compared to BM-MSC. CD34⁺ or TNC CB units were diluted in expansion media containing 20% FBS and 100 ng/ml G-CSF, SCF, and TPO. These cells were plated on a pre-established confluent layer of MSC. Co-culture occurred for 7 days at 37° C. in a humidified incubator. After 7 days the non-adherent cells were removed and replaced with fresh media and the plates were allowed to continue culture for an additional 7 days. On day 14, non-adherent cells were harvested separately from adherent cells and both populations were assayed for the presence of CD34⁺CD38⁻ cells, progenitor colony forming cells, LTCIC cells, and transplantable HSC.

The numbers of CD34⁺CD38⁻ phenotypically defined HSC were assessed pre- and post-expansion by multi-variant flow cytometry. Cells were stained with antibodies and no less than twenty thousand events were accumulated for each analysis. The staining protocol was as follows. Cells were first washed in PBS/penicillin/streptomycin/1% BSA and resuspended in 100 μl of PBS/penicillin/streptomycin/1% BSA containing the appropriate antibodies for 1×10⁶ cells. Samples were then mixed and incubated at 4° C. in the dark for 40 min. The cells were then washed twice in PBS/penicillin/streptomycin/1% BSA and fixed in PBS/1% paraformaldehyde for later flow cytometric analysis. For cell sorting cells were not fixed and were sorted immediately into fresh media. Data is presented as both the % of cells positive as compared to negative background staining (isotype) control and as changes in mean fluorescence intensity.

A progenitor assay evaluates the proliferative, survival, and differentiation potential of cells by progenitor colony assay. MNC or CD34 cells were plated in triplicate with growth factors for colony formation by granulocyte macrophage colony-forming units (CFU-GM), erythroid blast-forming units (BFU-E), and granulocyte macrophage, erythroid, and megakaryocytic colony-forming units (CFU-GEMM). Human cells (100,000) were plated for colony formation in 1% methylcellulose culture medium with 30% FBS, 1 U/ml recombinant human erythropoietin (Epo), 100 U/ml recombinant human granulocyte macrophage-colony stimulating factor (GM-CSF), 100 U/ml recombinant human Interleukin-3 (IL-3), and 50 ng/ml recombinant human Stem Cell Factor (SCF, steel factor, SCF). Cells were scored after 14-day incubation at 5% CO₂ and 5% O₂.

Long-term culture-initiating cell (LTCIC) assays were performed to more accurately assess clonogenic cells. Confluent irradiated feeder layers of bone marrow stromal cells (from normal donors) were established and then seeded with the test samples at a known concentration, incubated and fed regularly with fresh medium. After 35 days, each well was harvested of non-adherent and adherent cells which were then assayed for CFU-GM as described herein. The number of LTCIC in each culture was then calculated by dividing the total number of colonies by four. LTCIC frequency was measured at 5 weeks by limiting dilution and the percentage of CD34⁺ cell population was determined. Prior studies have demonstrated that one LTCIC will produce four clonogenic cells after a 5-week culture.

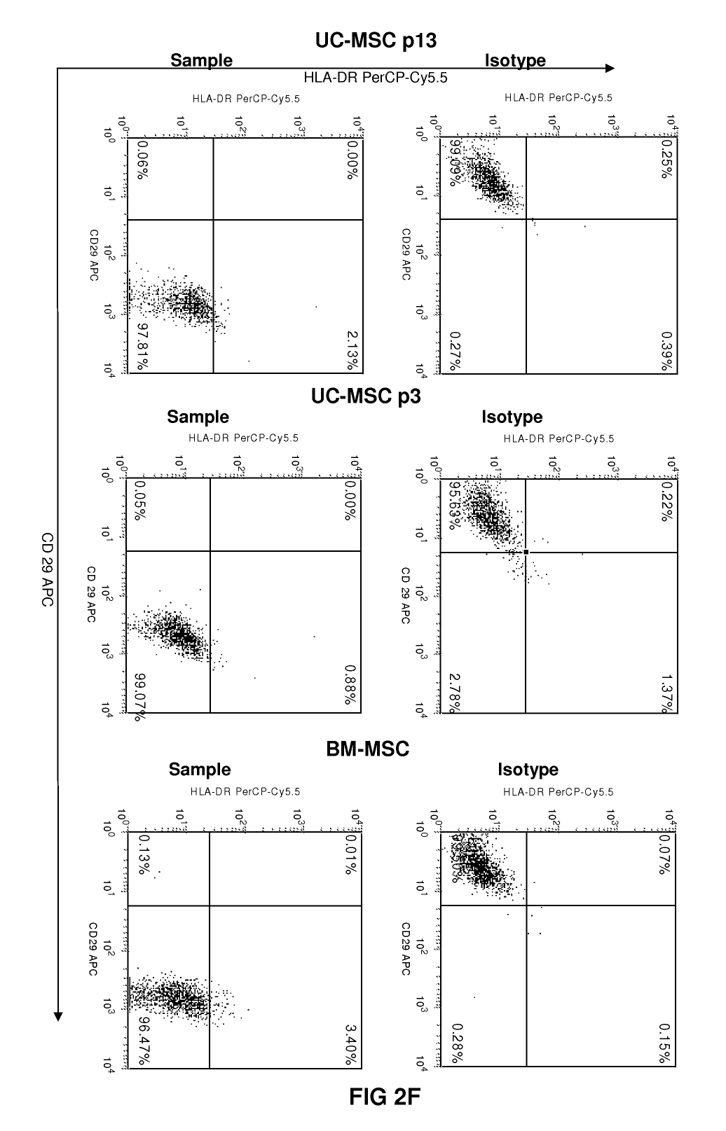

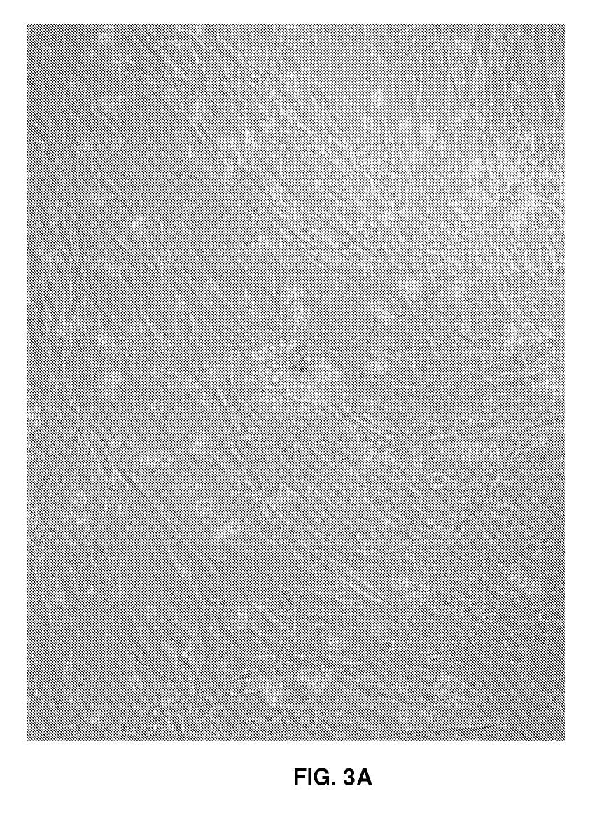

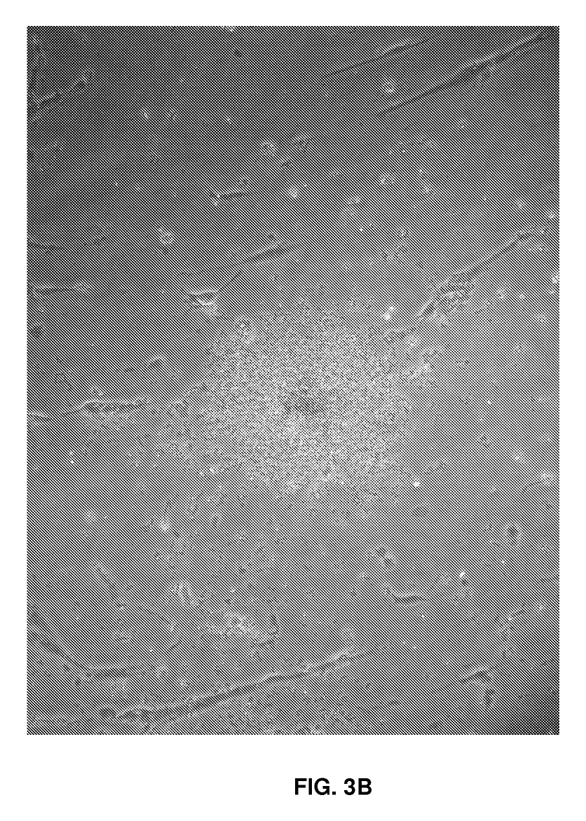

FIG. 3 demonstrates the results of UC-MSC ability to support the growth of pooled CD34⁺ cord blood cells in long term culture-initiating cell (LTC-IC) assays, as compared to BM-MSC. Like BM-MSC, CB-MSC express a defined set of cell surface markers. Flow cytometry demonstrates that both UC-MSC and BM-MSC are positive for CD29, CD44, CD73, CD90, CD105, CD166, HLA-A and negative for CD45, CD34, CD38, CD117, HLA-DR expression.

Utilizing Mitomycin C treated (100-200 μg/ml, 15 min.) or alternatively irradiated (15-20 Gy) UC-MSC from multiple donors as a feeder layer, it was observed that UC-MSC have the ability to support the maintenance of long term hematopoiesis during the LTC-IC assay (FIG. 3). Thus, umbilical cord MSC have the ability to support long term maintenance of hematopoietic stem cells as defined by the LTC-IC assay. Specifically, UC-MSC isolated from separate umbilical cord donors support the growth of 69.6±11.9 (1A), 31.7±3.9 (2B), 67.0±13.5 (3A), and 38.5±13.7 (3B) colony forming cells (CFC) per 1×10⁴ CD34⁺ cord blood cells as compared to 64.0±4.2 CFC per 1×10⁴ CD34⁺ cord blood cells supported by BM-MSC (mean ±SEM, N=4 separate segments from three different donors). Thus, umbilical cord derived Mesenchymal Stem Cells, a recently described novel source of MSC, have the ability to support long term maintenance of hematopoietic stem cells, as defined by the LTC-IC assay. This demonstrates that UC-MSC have utility for ex vivo expansion of cord blood derived HSC from the same donor or as an in vivo cellular therapy for hematologic diseases.

The usage of umbilical cord Mesenchymal Stem Cells in the hematology transplant setting can improve clinical outcome, for example through enhanced or expedited recovery of the blood or immune system post transplant for patients in need of cellular therapy as a result of the failure of their hematopoietic system. The failure may result from the development of hematologic diseases in response to exposure to environmental toxins, as a result of genetic abnormalities, as a result of the development of cancer, as a result of other past or ongoing medical intervention, or through an unknown mechanism.

### Cell Morphology

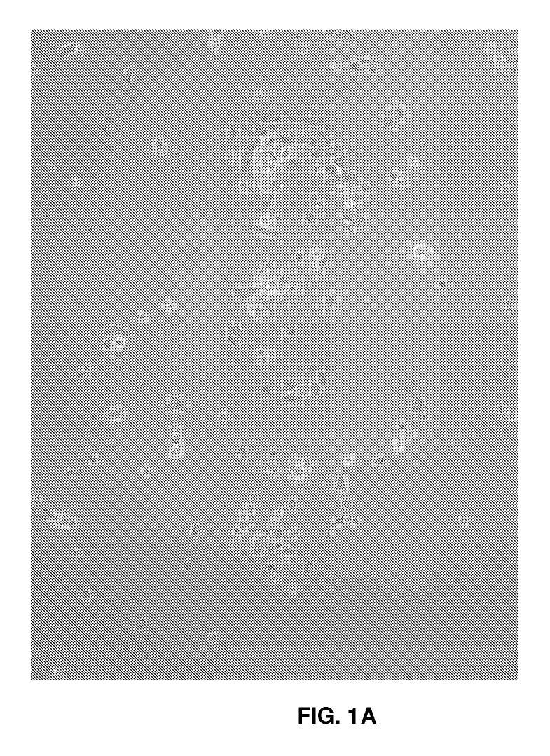

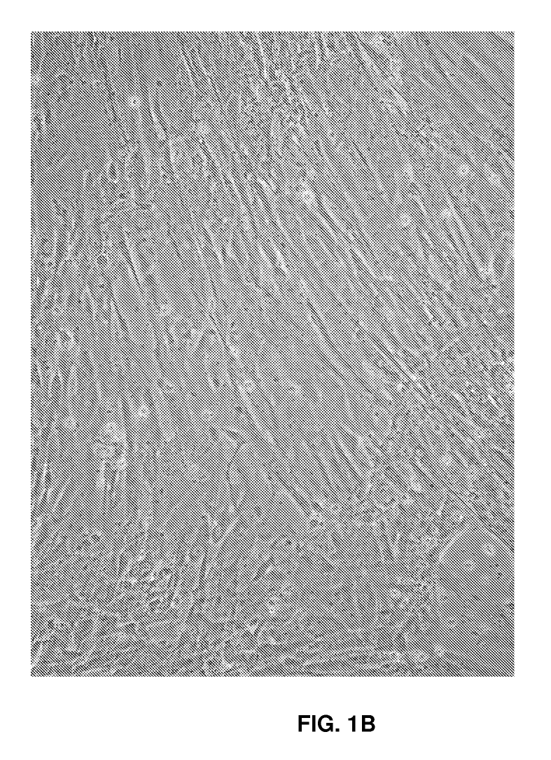

UC-MSC can be derived in a low glucose DMEM based media supplemented with serum and antibiotics. Under these conditions, UC-MSC display an adherent cell morphology and grow in colonies (FIG. 1A). Cells can be detached and re-plated to form a monolayer (FIG. 1B) similar to BM-MSC. However, UC-MSC can be cultured longer than BM-MSC, having the ability to be divided greater than 13 passages.

### Flow Cytometric Analysis

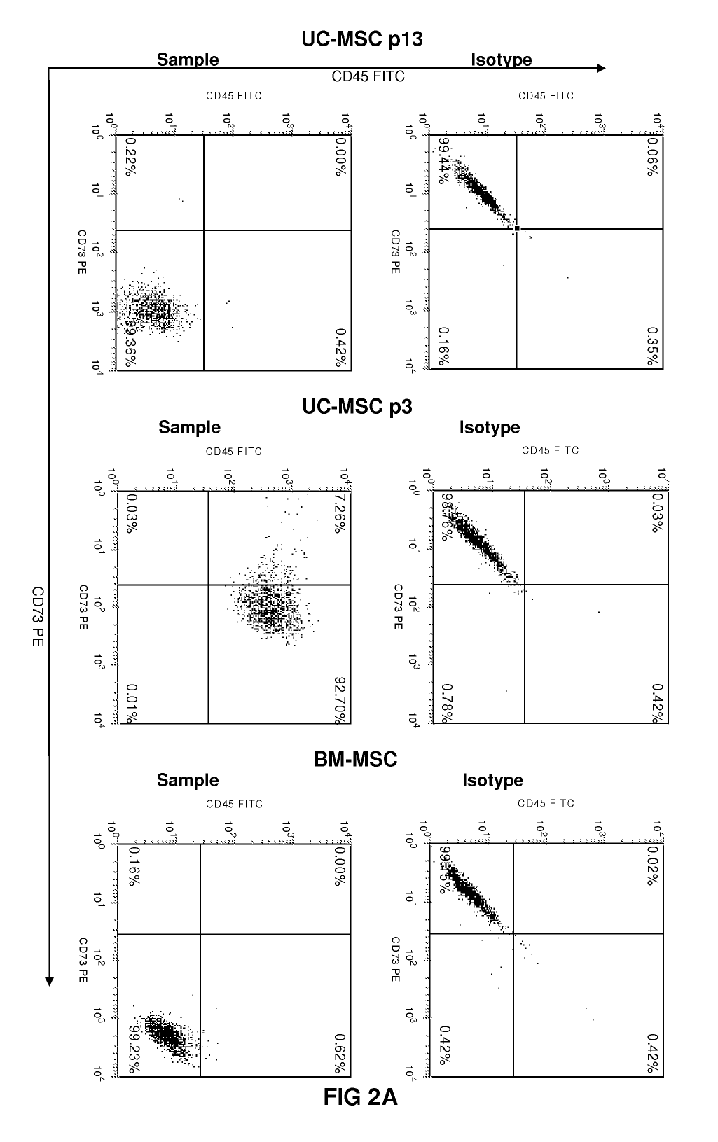

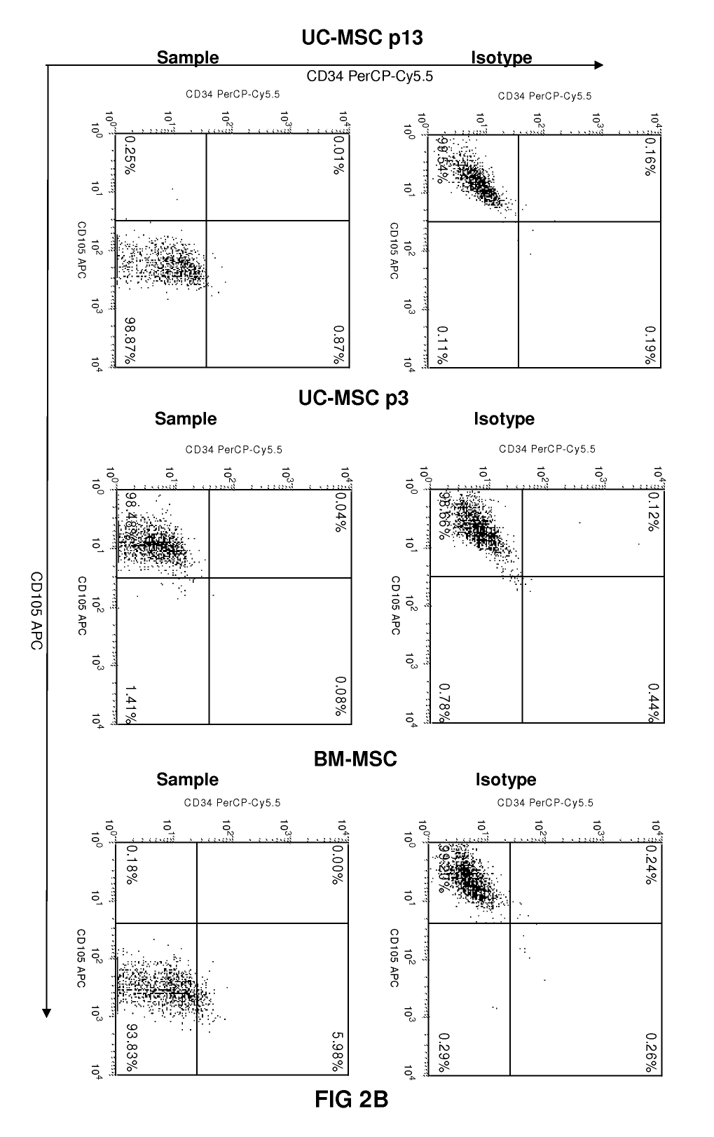

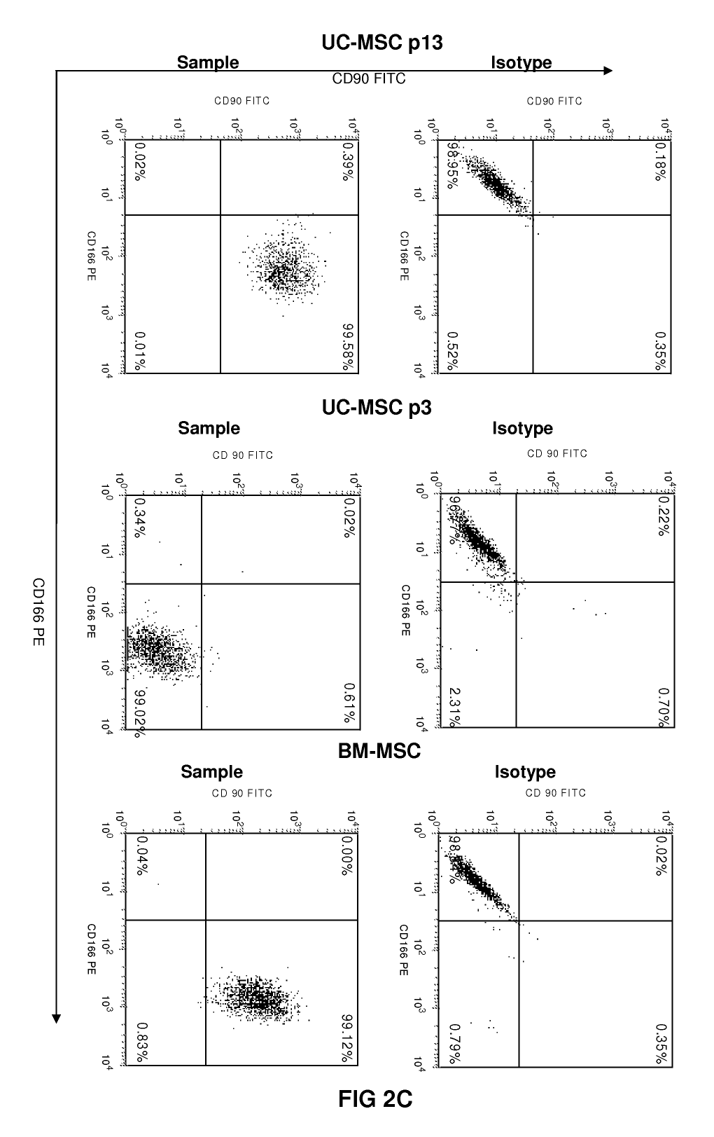

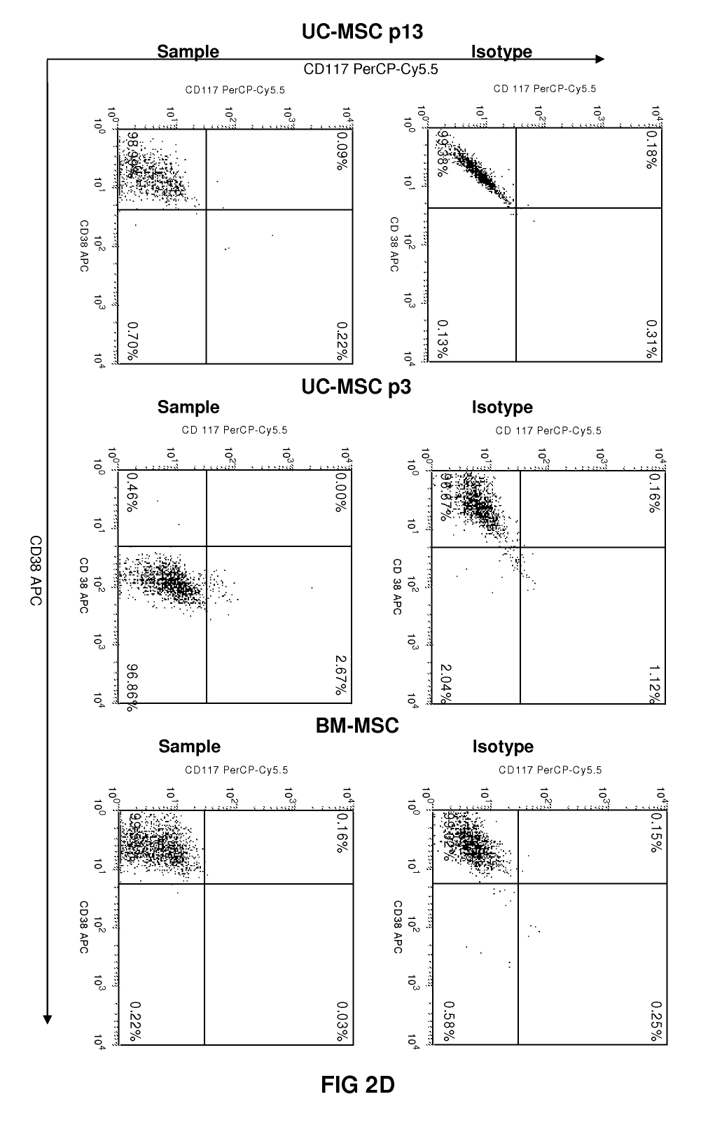

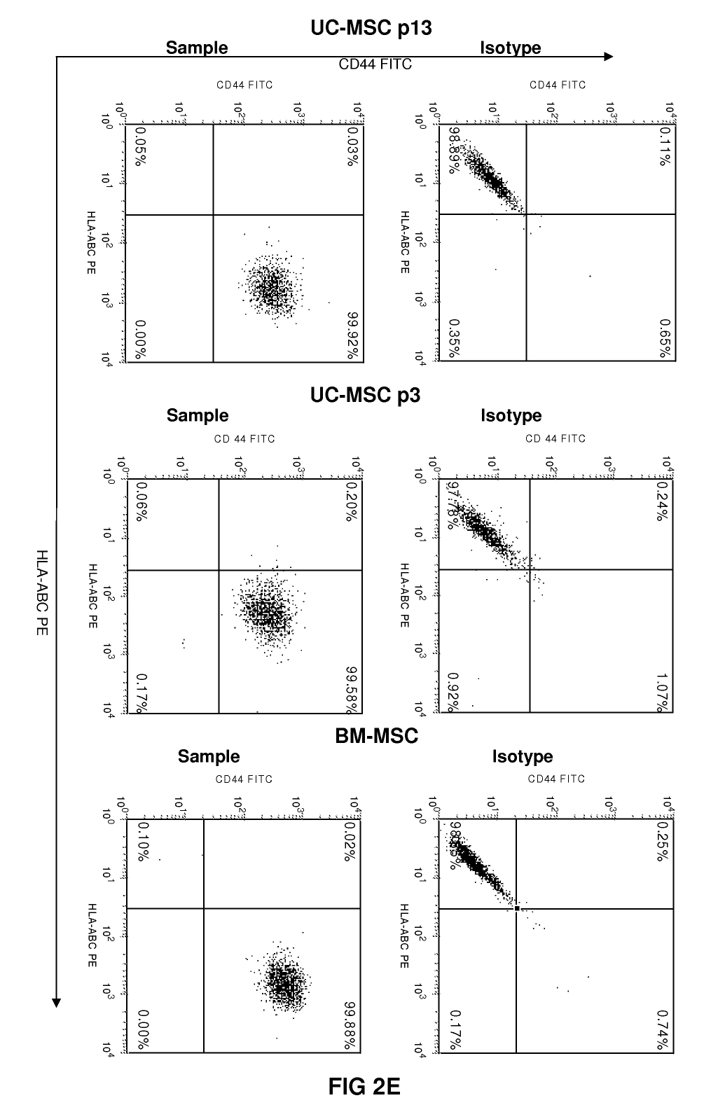

Both UC-MSC that have been cultured to passage 5 and BM-MSC were positive for CD29 (Ontegrin β1), CD44, CD73, CD90 (Thy-1), CD105 (Endoglin), CD166 (ALCAM), and HLA-A. In addition, both UC-MSC and BM-MSC were negative for CD45, CD34, CD38, CD117 (c-kit), and HLA-DR expression (Table 1). This expression profile is indicative of MSC, based on examination of BM-MSC cultures. Earlier passages of UC-MSC, however, have a slightly different profile (Table 1, FIG. 2). Specifically, passage 3 UC-MSC within the same culture dish have variable CD45 expression, express CD38, and lack CD105 and CD90.

### Long Term Culture-Initiating Cell Assay

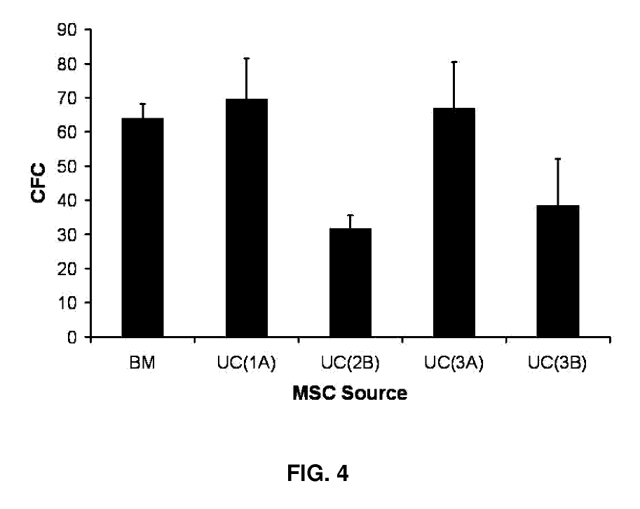

In order to test the putative UC-MSC in a manner relevant to hematopoietic function, their capacity to act as a feeder layer by supporting the growth and maintenance of HSC was evaluated. As a component of this experiment, CD34 cord blood cells were cultured on top of a UC-MSC feeder layer for 35 days and allowed to form colonies (FIG. 3A). The hematopoietic cells were then re-plated for an additional 14 days of culture in methocellulose supplemented with growth factors for hematopoietic progenitor colony formation (FIG. 3B). UC-MSC isolated from separate umbilical cord donors (numbered 1 through 3) from different sections of the cord (lettered A or B) support the growth of 69.6±11.9 (1A), 31.7±3.9 (2B), 67.0±13.5 (3A), and 38.5±13.7 (3B) colony forming cells (CFC) per 1×10⁴ CD34⁺ cord blood cells, as compared to 64.0±4.2 CFC supported by a BM-MSC stromal layer (Mean ±SEM, N=4 separate segments from three different donors) (FIG. 4). When the method of calculating LTC-IC frequency for bone marrow cells described by Sutherland et al. is applied to this situation, an LTC-IC frequency in the four umbilical cord sections of 17.4, 7.9, 16.7, 9.6 clonogenic cells per 1×10⁴ CD34⁺ cells is obtained utilizing UC-MSC as a feeder layer, and a LTC-IC frequency of 16.0 is obtained utilizing BM-MSC as a feeder layer.

One embodiment of the technology allows for co-cultured umbilical cord MSC and cord blood, which contains HSC, to then be transplanted into a recipient patient in a manner consistent with conventional hematopoietic stem cell transplantation procedures, meaning that cells are infused into the recipient by intravenous (IV) injection. As an alternative, intra-bone marrow injection as a delivery methodology may also be considered as a delivery strategy.

Target dosing of cells is expected to be 2×10⁶ umbilical cord MSC per kg body weight of the recipient. Dosing of umbilical cord MSC may however be adjusted based on variation from a minimal dosing of cord blood, which is expected to be the equivalent of 2×10⁶ CD34⁺ cells per kg body weight of the recipient. Alternatively, dosing of umbilical cord MSC could be calculated such that it is roughly equivalent to the CD34⁺ HSC dosing per kg body weight of the recipient. Permutations of this dosing in which the umbilical cord MSC dosing is higher or lower than the cord blood CD34+ dosing are also envisioned under patient specific, donor specific, and/or disease specific situations.

The preferred buffer for infusion is either normal saline (0.9% NaCl), Dextran 40 (10% Gentran 40 in 0.9% NaCl,)/Human Serum Albumin (HSA, 5% Buminate), or equivalent with or without 10% DMSO or other cryoprotectant. The preferred umbilical cord MSC concentration for infusion is expected to be 2.5×10⁶ per ml. The preferred time of infusion is 4-6 ml per minute.

Co-cultured umbilical cord MSC and cord blood may be either unfrozen or previously cryogenically preserved together or individually. The umbilical cord MSC may be infused either simultaneously or sequentially with the cord blood. Sequential injection of umbilical cord MSC may occur by infusion prior to or following infusion of the cord blood.

A further embodiment of the technology allows for the co-transplantation of umbilical cord MSC and cord blood in the absence of previous ex vivo co-culture. Under this scenario, the co-transplantation by infusion may be simultaneous or sequential. This co-transplantation of cells that have not been co-cultured ex vivo allows for the cells to interact following infusion thereby allowing for what can be viewed as in vivo co-culture.

Transplantation of umbilical cord MSC in conjunction with cord blood is envisioned to occur in both the myeloablative and non-myeloablative setting. Evaluation of subsequent transplantation outcomes, such as graft status, is expected to be performed by chimerism analysis. Chimerism can be evaluated utilizing various techniques including; cytogenetic analysis, fluorescent in situ hybridization (FISH), restriction fragment length polymorphism (RFLP), Microsatellite (STR) and minisatellite (VNTR) genotyping analysis, and real-time quantitative PCR. Ultimately, the optimal methodological approach chosen to detect mixed chimerism detection needs to be informative, sensitive, and quantitatively accurate.

### Materials and Methods

### Isolation of Umbilical Cord-Mesenchymal Stem Cells

Umbilical cord samples were obtained following the delivery of normal term babies with Institutional Review Board approval. A portion of the umbilical cord was then cut into approximately 3 cm long segments. The segments were then placed immediately into 25 ml of phosphate buffered saline without calcium and magnesium (PBS) and 1× antibiotics (100 U/ml penicillin, 100 ug/ml streptomycin, 0.025 ug/ml amphotericin B). The tubes were then brought to the lab for dissection within 6 hours. Each 3 cm umbilical cord segment was dissected longitudinally utilizing aseptic technique. The tissue was carefully undermined and the umbilical vein and both umbilical arteries were removed. The remaining segment was sutured inside out and incubated in 25 ml of PBS, 1× antibiotic, and 1 mg/ml of collagenase at room temperature. After 16-18 hours the remaining suture and connective tissue was removed and discarded. The cell suspension was separated equally into two tubes, the cells were washed 3× by diluting with PBS to yield a final volume of 50 ml per tube, and then centrifuged. Red blood cells were then lysed using a hypotonic solution. Cells were plated onto 6-well plates at a concentration of 5-20×10⁶ cells per well.

### Culture of Umbilical Cord-Mesenchymal Stem Cells

UC-MSC were cultured in low-glucose DMEM (Gibco) with 10% FBS (Hyclone), 2 mM L-Glutamine (Gibco), 100 U/ml penicillin, 100 ug/ml streptomycin, 0.025 ug/ml amphotericin B (Gibco). Cells were washed 48 hours after the initial plating with PBS and given fresh media. Cell culture media were subsequently changed twice a week through half media changes. After 7 days or approximately 70-80% confluence, cells were passed using HyQTase (Hyclone) into a 10 cm plate. Cells were then regularly passed 1:2 every 7 days or upon reaching 80% confluence. Alternatively, 0.25% HQ trypsin/EDTA (Hyclone) was used to passage cells in a similar manner.

### Cell Morphology

Cellular morphology was observed and documented under an inverted microscope and images were captured with a digital camera.

### Flow Cytometry

Flow cytometry was performed on putative UC-MSC in order to characterize the cells phenotypically and to compare them to BM-MSC. Monoclonal antibodies to specific human cell markers were utilized to stain cells and multivariate flow cytometry was utilized to analyze the surface expression. Cells were stained and analyzed in three antibody groups. Group A consisted of anti-human CD45-FITC, CD73-PE, CD34-PerCP-Cy5.5, and CD105-APC. Group B consisted of anti-human CD90-FITC, CD166-PE, CD117-PerCP-Cy5.5, and CD38-APC. Group C consisted of anti-human CD44-FITC, HLA-ABC-PE, HLA-DR-PerCP-Cy5.5, and CD29-APC. The staining protocol was as follows. The UC-MSC were detached from the plate using 2 ml of HyQtase (Hyclone) following a PBS wash and transferred to a 5 ml polystyrene tube. Cells were then washed with flow cytometry buffer (PBS/100 U/ml penicillin/100 ug/ml streptomycin penicillin/streptomycin/1% BSA) and resuspended at a concentration of 1×10⁶ cells/100 μl of buffer containing the appropriate antibodies. Samples were then mixed and incubated at 4° C. in the dark for 40 min. The cells were then washed twice with buffer and fixed in PBS/1% paraformaldehyde for later flow cytometric analysis.

### Isolation of Umbilical Cord-Hematopoietic Stem & Progenitor Cells

Umbilical cord blood samples were obtained with Institutional Review Board approval following the delivery of normal term babies. Mononuclear cells were obtained by density centrifugation on Ficoll-Paque PLUS (Amersham Biosciences). Phenotypically defined CD34⁺ enriched HSC/HPC were obtained from mononuclear cells by CD34 positive magnetic bead enrichment to a purity of greater than 98% on an AutoMACS (Miltenyi Biotech, Auburn, Calif.).

### Long Term Culture-Initiating Cell (LTC-IC) Assay

LTC-IC assays were performed to assess the ability of UC-MSC to support the growth and maintenance of cord blood-derived clonogenic cells using the following procedure. Confluent Mitomycin C (200 μg/ml)-treated feeder layers of UC-MSC or BM-MSC were established in six-well plates from initial plating at concentrations of 1×10⁵ cells per well. At 24 hours post-Mitomycin C treatment, each well was seeded with 1×10⁵ pooled CD34⁺ cord blood cells and incubated at 37° C. 5% CO₂ for 35 days. LTC-IC media consisted of IMDM, 20% FBS, 2 mM L-glutamine, 1000 units/ml Penicillin, 100 units/ml Streptomycin, and 1 μM hydrocortisone. Media were changed three times per week by half-media replacements. After 35 days, non-adherent and adherent hematopoietic cells were harvested and assayed for colony formation. Cells collected from each well were plated in triplicate for progenitor colony formation in 1% methylcellulose culture medium with 30% FBS, 1 U/ml recombinant human erythropoietin, 100 U/ml recombinant human granulocyte macrophage-colony stimulating factor (GM-CSF), 100 U/ml recombinant human Interleukin-3 (IL-3), and 50 ng/ml recombinant human Stem Cell Factor (SCF, steel factor). Cells were scored after a 14-day incubation at 5% CO₂ and 5% O₂. Data are presented as the absolute numbers of colony forming cells (CFC) per 1×10⁴ CD34⁺ cord blood cells.

### Tables

TABLE 1
Mesenchymal Stem Cell Surface Expression of Phenotypic Markers

| | | | | | | | | | | HLA- | HLA- | |
|---|---|---|---|---|---|---|---|---|---|---|---|---|
| | CD45 | CD73 | CD34 | CD106 | CD90 | CD166 | CD117 | CD38 | CD44 | ABC | DR | CD29 |
| BM-MSC p3 | − | + | − | + | + | + | − | − | + | + | − | + |
| UC-MSC p3 | +/− | + | − | − | − | + | − | + | + | + | − | + |
| UC-MSC p5 | − | + | − | + | + | + | − | − | + | + | − | + |
| UC-MSC p13 | − | + | − | + | + | + | − | − | + | + | − | + |

We claim:

1. A method to culture hematopoietic cells, the method comprising:
(a) obtaining mesenchymal stem cells from Wharton's jelly of a mammalian umbilical cord; and
(b) co-culturing cord blood cells with the mesenchymal stem cells.

2. The method of claim 1 wherein the cord blood cells are hematopoietic stem cells.

3. The method of claim 2 wherein the hematopoietic stem cells are further defined as CD34⁺.

4. The method of claim 1, wherein the mesenchymal stem cells are genetically matched to the cord blood cells.

5. The method of claim 4, wherein the cells are matched for HLA genotype.

6. The method of claim 2, wherein the mesenchymal stem cells are from Wharton's jelly and the hematopoietic stem cells are from cord blood, and both types of cells are from the same umbilical cord.

7. The method of claim 1, wherein growth, viability and maintenance of hematopoietic cells are supported during clonal expansion and differentiation.

8. A method for treating a subject with a hematologic disease, in need thereof, the method comprising:
(a) combining umbilical cord mesenchymal stem cells with cord blood hematopoietic stem cells; and
(b) transplanting the combined cells of (a) into the subject.

9. The method of claim 8, wherein combining is by co-culturing the cells in vitro.

10. An in vitro culture of mesenchymal stem cells from Wharton's jelly and cord blood cells.

11. A transplant comprising mesenchymal stem cells from Wharton's jelly and hematopoietic stem cells.

12. The transplant of claim 11 wherein the hematopoietic stem cells are from cord blood.
